(19) United States
(12) Patent Application Publication
(10) Pub. No.: US 2004/0137081 A1
(43) Pub. Date: Jul. 15, 2004

(54) Attaining sexual wellness and health of the sexual vascular system with proanthocyanidins

(76) Inventors: Peter Rohdewald, Munster (DE); Victor Ferrari, Feutersoey (CH)

Correspondence Address: MINTZ LEVIN COHN FERRIS GLOVSKY & POPEO, 666 THIRD AVENUE, NEW YORK, NY, 10017, US

(21) Appl. No.: 10/685,677

(22) Filed: Oct. 15, 2003

Related U.S. Application Data
(60) Provisional application No. 60/439,737, filed on Jan. 13, 2003.

(51) Int. Cl. A61K 33/00; A61K 31/7048; A61K 31/198

(52) U.S. Cl. 424/718; 514/27; 514/456; 514/575

(57) ABSTRACT

Sexual wellness or sexual fitness is enhanced over time by administrating on a daily basis a source of proanthocyanidins and a source of arginine. Both sources may be blended into a composition or taken separately from a kit. The source of arginine may be a salt or peptide of L-arginine and aspartic acid such as arginine aspartate. The proanthocyanidins stimulate an endothelial NO-synthase enzyme, which serves as a catalyst for synthesis of the nitric oxide from a substration that is the source of the arginine. A sufficient amount of the nitric oxide is released over time to enhance sexual wellness or sexual fitness. In case of low levels of androgenic hormones in both sexes, the combination may contain as a further ingredient a sex hormone or a sex hormone precursor or a sex hormone stimulant or a sex hormone bioavailability enhancer.

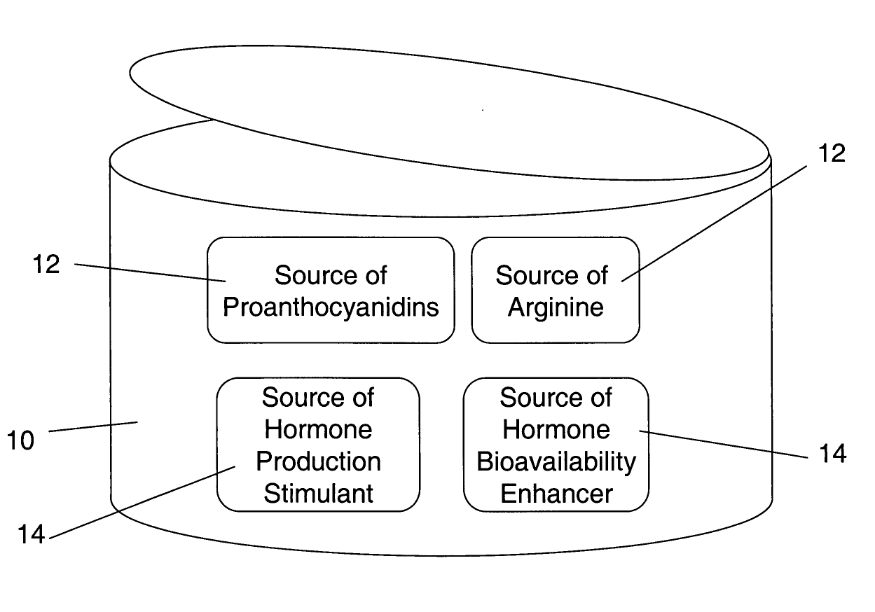

### CROSS-REFERENCE TO RELATED PATENT APPLICATION

This application claims priority from U.S. provisional application Serial No. 60/439,737 filed Jan. 13, 2003.

### BACKGROUND OF THE INVENTION

1. Field of the Invention

The invention relates to improving sexual fitness or wellness of both sexes and the health of the sexual vascular system with ingredients that include a source of proanthocyanidins, a source of arginine and, possibly, a hormone supplement. The source of proanthocyanidins may be a botanical extract and the source of arginine may be from arginine aspartate. The hormone supplement may be a hormone itself, a hormone precursor, a hormone production stimulator or a hormone bioavailability enhancer. All or some of the ingredients may be part of a composition and/or be separate from each other within a kit.

2. Discussion of Related Art

When a male is in his early twenties, it's easy to take peak sexual performance for granted. Yet as time passes, the male body's biological system changes, and he may notice that his sexual stamina, performance and even pleasure begin to decrease. Getting “in the mood” may start to take a little effort.

Many women have problems with sex when they reach menopause and their ovaries produce smaller amounts of sex hormones. Lower levels of estrogen can make the vaginal tissue dry, and less androgen leads to less sexual desire and arousal.

One important difference affecting sexual desire is that men have levels of testosterone that are 20 to 30 times what women have. Men's testosterone levels gradually decline over time but they do not experience a drop-off as women do at menopause. In men and women, testosterone and other androgens work to increase desire.

Androgen gels and patches for women are being considered for women with sexual dysfunction. Another possibility to overcome the female androgen deficiency syndrome is to supply women with 50 mg dehydroepiandrosterone per day, which facilitates the enhanced production of testosterone, dehydrotestosterone, androstenedione and androstenediol. That improved female androgenic profile causes intense sexual thoughts and a general enhancement in mental and physical sexual arousal (Spark, R. F., 2002; Hackbert, L. and Heiman J. R., 2002).

To increase blood flow in the female genitale tissue is also useful to improve sexual wellness. The New York Times on Mar. 25, 2003 published an article entitled “Effort to Make Sex Drug for Women Challenges Experts”. According to the article, researchers found that women's sex organs are not as readily affected as men's by sildenafil, which is the active ingredient of a drug sold under the trademark VIAGRA. Blocking the same enzyme in women that normally inhibits blood flow does not increase circulation to genital tissue so drastically as in men for causing engorgement of erectile tissue.

Studies suggest that sildenafil alone does not fix female arousal problems. However, when taken together with supplemental hormones, at least one study showed that 57 percent of 202 postmenopausal women involved in a study reported improved genital sensations, compared with 43 percent of a placebo group. Forty-one percent of the sildenafil group members reported greater satisfaction with sex, compared with 27 percent in the placebo group. Although the differences between the two groups were modest, the study suggests that sildenafil could help women with healthy hormone levels and in happy relationships.

One may surmise that female sexual function is accomplished physiologically in a similar manner like in man in a way that cGMP triggers lubrication and engorgement of the clitoral tissue. The studies mentioned in the previously mentioned article suggest the possibility that when women have a healthy hormone level, such dietary supplements may help improve sexual function in women to some extent. Another way to increase the blood flow into the female or male sexual organs is to increase the production of nitric oxide, which in turn triggers the release of cGMP. Whereas sildenafil and related substances lead to a sustained increase of blood content of the male or female sexual organs by blocking the enzymatic destruction of the vasodilating cGMP, nitric oxide produces the same increased blood volume by enhancing the production of cGMP.

As a physiological source for nitric oxide production, the aminoacid L-arginine is used. The enzyme endothelial nitric oxide synthase produces nitric oxide from L-arginine. To provide an enhanced and sustained blood flow to the sexual organs, it is first of all necessary to supplement the organism with the substrate L-arginine in sufficient quantities. However, the presence of high concentrations of L-arginine alone does not lead to a substantial higher blood flow to the sexual organs. It is necessary to stimulate additionally the endothelial nitric oxide synthase, so that nitric oxide production from L-arginine is catalyzed by the active enzyme. A potent stimulator of endothelial nitric oxide synthase is a proanthocyanidins-containing extract.

Proanthocyanidins represent a group of plant polyphenols found in roots, barks and fruits with an astringent taste. Proanthocyanidins include the subgroups of procyanidins and prodelphinidins. Proanthocyanidins are biopolymers composed of flavan subunits. Procyanidins are composed of catechin and epicatechin units, also called monomeric procyanidins.

Proanthocyanidins are extracted from plant material by conventional methods using solvents like water, ethanol or acetone or fluid carbon dioxide. The extracts are purified by solvent/solvent extraction, ultra filtration or chromatographic procedures. The purified extracts are concentrated by solvent evaporation, freeze drying or spray drying.

A proanthocyanidin-rich extract from the bark of French maritime pine is distributed under the tradename Pycnogenol® by Horphag Research, Switzerland. The extract contains 70-75% by weight proanthocyanidins and other flavanols such as catechin, epicatechin and taxifolin. Other proanthocyanidins rich extracts can be obtained from grape seeds, cones from cypress trees, cocoa beans or other plant materials. Pycnogenol®) pine bark extract has been shown to stimulate endothelial nitric oxide synthase and to induce vasodilation (Fitzpatrick, D. F., Bing, B., Rohdewald, P., 1998)

### SUMMARY OF THE INVENTION

One aspect of the invention resides in a product that, when administered, offers both sexes a safe, natural way to preserve and maintain sexual responsiveness, endurance and enjoyment. It includes a blend of ingredients, namely, proanthocyanidins and a substrate that is a source of arginine, preferably a salt or dipeptide of L-arginine and aspartic acid, such as arginine aspartate. When the blend is administered, the endothelial NO-synthase is stimulated by the proanthocyanidins. Nitric oxide is released from the substrate in response to the stimulated endothelial NO-synthase enzyme, which acts as a catalyst for synthesis of the nitric oxide from the substrate. The source of arginine and proanthocyanidins are in therapeutically effective amounts to cause a sufficient amount of the nitric oxide to be released from the synthesis so that when fresh supplies are taken on a daily basis over a period of time, sexual fitness or sexual wellness improves by the end of the period of time. The blend may contain testosterone or dehydroepiandrosterone to overcome the lack of androgenic hormones thereby increasing or restoring sexual arousal and desire.

### BRIEF DESCRIPTION OF THE DRAWING

The drawing shows a kit in accordance with the invention.

### DETAILED DESCRIPTION OF THE INVENTION

The invention pertains to the prolonged use of a blend of ingredients, namely, a source of arginine (such as L-arginine) and a source of proanthocyanidins. Preferably, the source is a salt or peptide of arginine and aspartic acid, namely, arginine aspartate. Preferably, the source of proanthocyanidins is derived from Pycnogenol®) or from other proanthocyanidin-containing extracts.

An oral administration of the blend in accordance with an administration regimen over a prolonged period of time provides certain benefits, which include helping to protect, restore and sustain blood vessel health and improve blood flow to the genital area, naturally enhancing male erections or female tumescence, naturally enhancing the body's sexual response and improving the health of the sexual vascular system.

By orally administering the blend of a source of arginine and a source of proanthocyanidins, the benefits to sexual fitness or sexual wellness are realized. That is, over time, the cumulative effect of the blend leaves one experiencing a heightened sense of sexual well-being.

The addition of androgenic hormones or precursors of androgenic hormones allows for an increase in sexual arousal and desire in case that production of androgenic hormones in women or men is insufficient to provide the mental prerequisite for sexual wellness. Some examples of precursors of androgenic hormones include dehydroepiandrosterone (DHEA) and dehydroepiandrosterone sulfate (DHEAS).

Herbalife commercializes two men's wellness products. One is a stimulator that is commercialized under the tradename OPTIMUM PERFORMANCE, which helps stimulate production of the male hormone testosterone to improve libido and enhance muscle development. The other is commercialized under the tradename MALE FACTOR 2000 to assist the testosterone to be more bioavailable and enhance vitality, stamina and muscle definition by increasing a proportion of testosterone in its free form. When these two wellness products are taken in combination with the blend of a source of arginine and a source of proanthocyanidins of the present invention, the men's sexual drive and wellness to attain added stamina, vigor, and performance.

While drugs for sexual enhancement may offer a temporary solution or tempting “quick fix,” they are associated with unwanted side effects and can be expensive. The blend according to the present invention, which is a natural dietary supplement, offers a safe, natural and cost-effective alternative.

The blend of the present invention may be in the form of a composition, taken either in tablet form or in liquid form. Alternatively, the blend may be in the form of the ingredients being in separate, distinct tablet or liquid form but packaged together in a kit 10. In the latter case, the separate ingredients 12 are taken either simultaneously, such as by mixing them together if in liquid form, or one after another if in tablet form. Additional ingredients 14 such as a hormone (testosterone or estrogen for example) or a hormone production stimulant, a source of a hormone (testosterone or estrogen, for example) bioavailability enhancer, a precursor of a hormone may be included in the Kit 10.

### EXAMPLE 1

Dr. Romil Stanislavov and V. Nikolova of The Seminological Laboratory SBALAG in Maichin Dom, Sofia, Bulgaria investigated the possibility to overcome erectile dysfunction by increasing the amounts of endogenous NO. For this purpose, Pycnogenol® pine bark extract as it is known to stimulate nitric oxide synthase was orally administered together with L-arginine as substrate for this enzyme.

The study included 40 men aged 25 to 45 years (mean age 36.6±5.3 years) with confirmed functional erectile dysfunction of at least 3 months duration, who were unable to achieve adequate erection and rigidity sufficient for vaginal penetration and completion of successful intercourse after spontaneous sexual stimulation. All patients were required to be involved in a stable, monogamous relationship with a female partner for more than 6 months.

Exclusion criteria were severe cardiovascular diseases, hypertension, renal failure, hepatic insufficiency, endocrine abnormalities, and psychiatric disorders. Patients were also excluded if they were currently or recently treated for erectile dysfunction by vasoactive medications, surgery, or any mechanical device. Concomitant use of other therapies for erectile dysfunction were not allowed. The study was approved by the Ethics Committee of Medical Research Board of the University Clinic of Sofia, Bulgaria and written consent was obtained from each participant.

All participants completed the questionnaire of O'Leary (O'Leary M P, et al. A brief male sexual function inventory for urology. Urology 1995; 46:697-706), which contains 11 questions addressing topics such as sexual drive, erectile function, problem assessment and overall sexual satisfaction. An additional sexual function questionnaire specially designed by us addressed the total number of attempts, and the total number of unsuccessful attempts with special emphasis on distinguishing between variants of unsatisfactory erections.

1. Weak ned: the penis is increasing in size and gains firmness to a certain extent which, however, is insufficient for penetrating the vagina.

2. Delayed: the erection is insufficient for penetration, yet the process requires more than the usual time.

3. Hesitating: before or during the contact the erection is unstable making the sexual intercourse difficult.

4. Loosing: during the love game an erection is achieved which, however, is lost while trying to penetrate or during intercourse.

During the study period all patients were asked to keep a sexual activity diary. The first three weeks of the study were carried out without medication as run-in phase in order to obtain reliable baseline values. All patients were treated for the first month with, three ampoules per day, of Sargenor (Sarget Pharma, Cedex, France). Each Sargenor ampoule contains 1 g L-arginine aspartate (equivalent to 0.57 g L-arginine) dissolved in 5 ml liquid. During the second month patients continued the Sargenor regimen and were additionally treated with 40 mg Pycnogenol® tablets (Hankintatukku, Helsinki, Finland) two times daily, in the morning and evening, respectively. During the third month patients continued using Sargenor and increased the Pycnogenol® extract dose to 3×40 mg per day. The response to treatment with Sargenor alone or in combination with Pycnogenol® was evaluated according to the accepted scale of assessing erection (O'Leary M P, et al. A brief male sexual function inventory for urology. Urology 1995; 46:697-706). The evaluations were made before and after each one-month stage of the treatment, using both the case history as well as the inquiry method. In order to ensure reliability of the data, the inquiry was filled out by the male patient, while the female partner filled out a short inquiry with questions raised by us.

The results were analyzed statistically, with the mean (SEM) and Student's t-test used for statistical comparison, with p<0.05 considered to indicate significance.

After treatment with Sargenor for one month only 2 patients, (5% of all patients) experienced normal erections (table 1). The improvement, however, did not reach significance over pre-treatment. The addition of 80 mg Pycnogenol® extract per day to the continued regimen of Sargenor after one month yielded a significant improvement, with 32 patients (80%) having normal erections. Another month treatment with Sargenor together with an increased amount of Pycnogenol® extract (120 mg per day) further improved the number of patients with recovered normal erectile function. At the end of the trial 37 patients, equivalent to 92.5% of all participants, were recovered.

TABLE 1
Clinical variants of disturbed erections of 40 patients prior to and post
treatment with Sargenor only (for 1 month) as well as combined
treatment with Sargenor and Pycnogenol ® for a second and third
month. Statistical significance of treatment outcome versus
pre-treatment is marked by an asterisk (p < 0.05), or two asterisks
(p < 0.01) (student's T-test).

| | | | | Sargenor + |
|---|---|---|---|---|
| Variant of | | | Sargenor + 80 mg | 120 mg |
| disturbed | Before | Sargenor | Pycnogenol ® | Pycnogenol ® |
| erection | treatment | (1. month) | (2. month) | (3. month) |
| Weakened | 22 (55%) | 20 (50%) | 5 (12.5%)* | 2 (5%)** |
| Delayed | 12 (30%) | 10 (25%) | 2 (5%)* | (0%)** |
| Hesitating | 2 (5%) | 4 (10%) | 1 (2.5%)* | 1 (2.5%)** |
| Loosing | 4 (10%) | 4 (10%) | 0 (0%)** | 0 (0%)** |
| Normal | 0 (0%) | 2 (5%) | 32 (80%)** | 37 (92.5%)** |

In those patients who gained normal erectile function during treatment, the time until erection developed in response to spontaneous sexual stimulation as well as the duration of the erection was recorded (table 2). The two patients responding to treatment with Sargenor only, required 10 minutes for the response to emerge. The combined treatment with Sargenor plus Pycnogenol® extract dramatically reduced the required time. While the mean duration of the erection was rather short when patients were treated with Sargenor only, it doubled in response to addition of 80 mg Pycnogenol® extract per day. Increasing the dosage of Pycnogenol® extract to 120 mg per day dramatically prolonged duration of the erection (table 2).

TABLE 2
The main characteristics of erectile response of patients who
experienced restored erectile function. Given is the mean time
(SEM) until erection developed in response to spontaneous sexual
stimulation, as well as the duration, of the erection.

| | | | Sargenor plus | Sargenor plus |
|---|---|---|---|---|
| | | | Pycnogenol ® | Pycnogenol ® |
| | Before | Sargenor | (80 mg/d) | 120 mg/d) |
| | treatment | (1. month) | (2. month) | (3. month) |
| Responders | 0 (0%) | 2 (5%) | 32 (80%) | 37 (92.5%) |
| Mean time | — | 10 ± 2 min | 4 ± 1 min | 2 ± 1 min |
| until | | | | |
| response | | | | |
| emerges | | | | |
| Mean | — | 2 ± 1 min | 4 ± 1 min | 15 ± 3 min |
| time of | | | | |
| duration | | | | |

Treatment with Sargenor in combination with Pycnogenol® extract is effective irrespective of the age of the person and the aetiology of the erectile dysfunction. Furthermore, the outcome was favorable independent of the duration and previous treatment of the erectile dysfunction.

During the treatment no side effects were observed. The patients did not report hyperstimulation and priapism was not observed.

However, in documented clinical experience with 2000 Pycnogenol® treated patients the rate of unwanted effects was 1.5%, consisting primarily of gastrointestinal disturbances and rare cases of dizziness, nausea and headache. The majority of unwanted effects were minor in nature (Rohdewald P. French maritime pine bark extract (Pycnogenol®), a versatile herbal supplement. Clin Pharmacol Ther., 2002).

This study demonstrates that it possible to effectively overcome functional erectile dysfunction in a natural way. Both, L-arginine and the components of Pycnogenol® pine bark extract, (procyanidins, catechin, taxifolin and phenolic fruit acids), are natural dietary constituents. Prescribed drugs which inhibit phosphodiesterase type 5 (PDE 5) are frequently applied “on demand” and the desired action requires 30-60 minutes (Uckert S. Kuthe A, Stief C G, Jonas U. Phosphodiesterase isoenzymes as pharmacological targets in the treatment of male erectile dysfunction. World J Urol 2001;19:14-22, Meulemann E, et al. A dose-escalation study to assess the efficacy and safety of sildenafil citrate in men with erectile dysfunction. BJU Int 2001;87:75-81).

Furthermore, PDE 5 is not exclusively located in the penis, and a broad range of side effects is the result: headache, nausea, flushing, rhinitis, dyspepsia and dizziness. Most disturbing are actions on the retina resulting in altered color perception and abnormal vision described as “star vision” (Meulemann E, et al. A dose-escalation study to assess the efficacy and safety of sildenafil citrate in men with erectile dysfunction. BJU Int 2001;87:75-81). A considerable number of acute adverse effects have been linked to sildenafil use, including syncope, myocardial infarction, cerebrovascular events, and even fatal cardiovascular events (Cohen J S. Is the sildenafil product information adequate to facilitate informed therapeutic decisions. Ann Pharmacother 2001;35:337-342). In view of the mechanisms of action it is suggested to be cautious using sildenafil together with Pycnogenol®). While Pycnogenol®) enhances production of NO which in turn triggers production of the second messenger cGMP, sildenafil inhibits degradation of the latter.

Therefore, both substances acting in concert might cause escalating up-regulation of cGMP. A synergistic effect with Pycnogenol® extract may allow a dose reduction of sildenafil to achieve the same benefit with less side effects:

### EXAMPLE 2

In the department of Dr. Stanislavov and Dr. Nikolova 50 men aged between 45-60 with testosterone levels below normal were treated daily with

Pycnogenol® 120 mg

L-arginine (Sargenor) 1,71 g

Testosterone undecanoate 120 mg

over a period of 12 months because of secondary infertility. The patients filled a questionnaire (Global efficacy questionnaire) whether the treatment improved the erection.

Before treatment, only 10% of the patients reported a normal erection.

TABLE 3
Assessment of efficacy and satisfaction of treatment

| Assessment | | After 1 month | After 3 months | After 6 months | After 12 months |
|---|---|---|---|---|---|
| of | Before | treatment with | treatment added | treatment added | treatment added |
| erection | treatment | L-arginine (only) | Pycnogenol ® + Andriol¹ | Pyonogenol ® + Andriol¹ | Pycnogenol ® + Andriol¹ |
| Disturbed: | 45 | 42 (84%) NS | 30 (60%)* | 21 (42%)* | 12 (24%)** |
| | (90%) | | | | |
| weakened | | | | | |
| delayed | | | | | |
| hesitating | | | | | |
| loosing | | | | | |
| Normal | 5 (10%) | 8 (16%) | 20 (40%)* | 29 (58%)* | 38 (76%)** |

¹Andriol: Testosterone undecanoate 40 mg (Organon GmbH);
NS - non significant;
*p < 0.05;
** p < 0.01

After 1 month treatment with L-arginine, the number of men with a normal erection increased non-significantly to 16%. After addition of Pycnogenol® and testosterone undecanoate to treatment, the percentage of men experiencing a normal erectile function increased from 40% up t 76% over the treatment period. In addition, the quality of sperm was significantly improved and 44% of the couples achieved pregnancy.

Example 2 demonstrates that men with abnormally low levels of testosterone and subsequent secondary infertility and disturbed sexual function greatly improved in terms of erectile function as well as in quality of sperms. The continuous supplementation with L-arginine together with the stimulator of endothelial nitric oxide synthase and the sex hormone testosterone was successful in improving sexual wellness and fertility.

The supplementation with L-arginine and a proanthocyanidin-containing extract on a regular basis allows healthy couples to spontaneously react to their partners stimulation. The combined supplementation with an added hormone, hormone precursor or hormone bioavailability enhancer is very useful for both sexes in case of reduced production of androgenic hormones.

After attaining the optimum level of sexual wellness and health of the sexual vascular system in accordance with the invention, missing one daily administration of the ingredients of the composition or within the kit of the invention will not diminish the level of sexual wellness and health of the sexual vascular back to the same level as it was prior to the taking of the present invention the first time. There will be some lingering, residual effect in the body that will carry over to the following day so that a daily administration of a single dose or serving thereafter will eventually restore the sexual wellness and health of the sexual vascular system to the same optimum level as before.

While the foregoing description and drawings represent the preferred embodiments of the present invention, it will be understood that various changes and modifications may be made without departing from the spirit and scope of the present invention.

What is claimed is:

1. A composition to enhance a level of sexual wellness for both sexes, comprising ingredients that include:
a substrate as a source of arginine and, subsequently, for nitric oxide; and
a stimulator that includes proanthocyanidins in an amount to stimulate an endothelial NO-synthase enzyme, which serves as a catalyst for synthesis of the nitric oxide;
the ingredients being in therapeutically effective amounts so that, when the ingredients are administered at least daily over a period of time, a sufficient amount of the nitric oxide releases from the synthesis to enhance a level of sexual wellness by an end of the period of time.

2. A kit comprising a container with ingredients that include:
a substrate as a source of arginine and, subsequently, for nitric oxide; and
a stimulator that includes proanthocyanidins to stimulate an endothelial NO-synthase enzyme, which serves as a catalyst for synthesis of the nitric oxide;
the ingredients being in therapeutically effective amounts so that, when the ingredients are administered at least daily over a period of time, a sufficient amount of the nitric oxide releases from the synthesis to enhance a level of sexual wellness by an end of the period of time.

3. A kit of claim 2, further comprising at least a hormone supplement within the container, the hormone supplement being selected from a group consisting of a hormone concentration increaser that, when administered, increases concentration of hormones in blood, a hormone production stimulate that, when administered, stimulates production of hormones, and a precursor of hormones.

4. A kit of claim 2, further comprising at least one hormone supplement within the container that, when administered, increases a proportion of a hormone in its free from to be more bioavailable.

5. A kit of claim 2, further comprising at least one hormone supplement within the container, the hormone supplement having an ingredient selected from a group consisting of the sex hormone, a precursor of the sex hormone and a stimulator of a production agent of the sex hormone configured to increase bioavailability of a sex hormone upon administration.

6. A kit of claim 2, wherein the substrate and the stimulator are part of a composition.

7. A method of attaining enhanced sexual wellness by stimulating nitric oxide synthase enzyme and releasing nitric oxide, comprising:
administering a composition daily over a period of time, the composition comprising ingredients that include a substrate and a stimulator, the substrate being a source of arginine and, subsequently, of nitric oxide; the stimulator including proanthocyanidins to stimulate an endothelial NO-synthase enzyme, which serves as a catalyst for synthesis of the nitric oxide; and
releasing a sufficient amount of nitric oxide from the synthesis in response to the administering to enhance sexual wellness by an end of the period of time.

8. The method of claim 7, wherein the administering includes initially administering an elevated dosage of the composition to attain the enhanced level of sexual wellness by the end of the period of time and thereafter administering a dosage of the composition daily that contains less of the composition than the elevated dosage and still provide the enhanced level of sexual wellness.

9. A method of claim 7, further comprising administering additionally a hormone supplement, the hormone supplement being selected from a group consisting of a hormone concentration increaser that, when administered, increases concentration of hormones in blood, a hormone production stimulator that, when administered, stimulates production of hormones, and a precursor of hormones.

10. A method of claim 7, further comprising administering at least one hormone supplement increases a proportion of a hormone in its free from to be more bioavailable.

11. A method of claim 7, further comprising administering a hormone supplement having an ingredient selected from a group consisting of the sex hormone, a precursor of the sex hormone and a stimulator of a production agent of the sex hormone configured to increase bioavailability of the sex hormone upon administration.

12. A method of attaining enhanced sexual wellness by stimulating nitric oxide synthase enzyme and releasing nitric oxide, comprising:
administering supplies of ingredients at least daily over a period of time, the ingredients including a substrate that is a source of arginine and, subsequently, of nitric oxide, and including a stimulator that has proanthocyanidins in an amount sufficient to stimulate an endothelial NO-synthase enzyme, which serves as a catalyst for synthesis of the nitric oxide; and
releasing a sufficient amount of the nitric oxide from the synthesis in response to the administering to attain an enhanced level of sexual wellness by an end of the period of time.

13. The method of claim 12, wherein the administering includes initially administering an elevated dosage of the composition to attain the enhanced level of sexual wellness by the end of the period of time and thereafter administering a dosage of the composition daily that contains less of the composition than the elevated dosage and still provide the enhanced level of sexual wellness.

14. A method of claim 12, further comprising administering a hormone supplement configured to increase bioavailability of a sex hormone upon administration, the hormone supplement having an ingredient selected from a group consisting of the sex hormone, a precursor of the sex hormone and a stimulator of a production agent of the sex hormone.

15. A method of claim 12, further comprising administering a hormone supplement having an ingredient selected from a group consisting of the sex hormone, a precursor of the sex hormone and a stimulator of a production agent of the sex hormone configured to increase bioavailability of the sex hormone upon administration.
